(19) United States
(12) Patent Application Publication
(10) Pub. No.: US 2015/0253224 A1
(43) Pub. Date: Sep. 10, 2015

(54) SUBSTRATE FOR SAMPLE HANDLING

(71) Applicants: STANLEY M. LIFFMANN, Newbury, MA (US); Peter Domenicali, Box Elder, SD (US)

(72) Inventors: STANLEY M. LIFFMANN, Newbury, MA (US); Peter Domenicali, Box Elder, SD (US)

(21) Appl. No.: 14/196,234

(22) Filed: Mar. 4, 2014

(51) Int. Cl. G01N 1/31; G01N 15/14; G01N 33/483; G01N 33/569; G02B 21/00; G01N 1/30

(52) U.S. Cl. CPC G01N 1/312; G02B 21/0096; G01N 1/30; G01N 33/4833; G01N 33/56972; G01N 15/1463; G01N 15/1425; G01N 2015/1486

(57) ABSTRACT

An automated microscopy system having a sample applicator configured to dispense a sample, a flexible ribbon having a surface configured to receive the sample, a light receiver, such as, for example, an automated microscope, and a ribbon controller configured to receive the flexible ribbon and guide the ribbon from the sample applicator to the light receiver. A monolayer of cells can be formed on a hydrophilic portion of the flexible ribbon and can be transported using the ribbon controller to the light receiver for analysis. The cell monolayer can be continuous.

### CROSS REFERENCE TO RELATED APPLICATIONS

This application claims priority to U.S. Provisional Application No. 61/772,247 filed Mar. 4, 2013, and U.S. Provisional Application No. 61/900,446 filed Nov. 6, 2013, the contents of each of which are incorporated herein by reference in their entireties.

### FIELD OF INVENTION

This invention relates generally to automated microscopy systems and methods, which may include depositing samples to be analyzed onto a substrate comprising a flexible ribbon or glass. In particular, some embodiments relate to forming a cell monolayer on the flexible ribbon or glass and transporting the cell monolayer to an automated microscope for analysis.

### BACKGROUND

Automated evaluation of biological samples is used in a large number of various medical, diagnostic, forensic and scientific applications. Microscopic evaluation of samples is evolving as the speed and sensitivity of digital cameras increase and the capability of computing devices processing and storing data acquired from biological sample analysis is steadily improving. Pattern recognition techniques may be used to process data acquired by microscopic and other analyses to thus analyze and classify different cell types. As the importance of imaging of biological samples and image analysis becomes established in research and clinical settings, there is a continuing need for rapid throughput, low cost, automated cellular microscopy systems.

Automated cellular analysis in the laboratory has followed two divergent paths. The first involved the use of automated microscopes, e.g., to enumerate, classify, and identify abnormal morphology associated with disease. The second involved the use of flow cytometers and electronic counters.

Microscopic classification of blood cells and analysis of their morphology is labor intensive, even when automated. Moreover, it may be challenging to prepare a sample to be analyzed having a desired quality. Use of flow cytometers may be time and labor intensive. Furthermore, flow cytometers require skilled operators and have significant acquisition, service, and maintenance costs.

Accordingly, there is a need for systems and methods that overcome the problems as discussed above.

### SUMMARY

In one embodiment, a substrate is provided having an optically clear, flexible ribbon or glass slide that may be utilized in automated microscopy systems. The flexible ribbon can have a hydrophilic surface and can be used as a substrate for a monolayer of cells. The monolayer may be continuous, which may greatly improve the quality of analysis of the sample using an automated microscope.

Use of the flexible ribbon can provide a number of advantages. For example, the flexible ribbon may allow more flexible positioning to the dispensing tip, a more hydrophilic surface, which may allow the formation of a continuous cell monolayer. The flexible ribbon may be cut into separate pieces, or portions, of any desired length. The separate portions of the ribbon may be transported for simultaneous processing at respective stations. Although more limited in application glass may be used instead of the flexible ribbon when a pathologist or hematologist would want to examine the stained monolayer manually. An example would be when performing a 5 part differential.

In some embodiments, a width of a cell monolayer may be controlled such that a single pass or multiple passes (which may be done at different magnifications) through an optical axis of a microscope may encompass all or one or more portions of the cells disposed on the ribbon.

In other aspects, a microscopy system is provided having a sample applicator with a tip end configured to dispense a sample, a flexible ribbon or glass having a hydrophilic surface configured to receive a sample from the tip, a light receiver, and a ribbon controller configured to receive the flexible ribbon and guide the flexible ribbon under the light receiver. In one embodiment, the sample applicator is configured to dispense a monolayer of cells on the hydrophilic surface of the flexible ribbon.

Methods for analyzing samples, e.g., cells, are also provided and can include engaging a flexible ribbon having a hydrophilic surface with a ribbon controller, dispensing cells from a tip of a sample applicator onto the hydrophilic surface; and guiding the ribbon under a light receiver with the ribbon controller, the light receiver analyzing the cells. In one embodiment, the ribbon may be guided under the light receiver at a substantially constant velocity.

In other aspects, a kit is provided having a flexible ribbon with a hydrophilic surface configured to receive a biological sample. The flexible ribbon can be formed from a variety of materials including, for example, polyester, polystyrene, mixtures thereof, and any other materials. The flexible ribbon can be optically clear in the range of about 400 nm to 700 nm. Furthermore, the kit can include instructions for installing the ribbon on a microscopy system, such as a microscopy system having a sample applicator with a tip end, a light receiver, a staining area, and at least one ribbon controller to receive and guide the ribbon from the sample applicator to the light receiver.

In one embodiment, the flexible ribbon can be formed from polymer (e.g., polyester, polystyrene, or any other material), or a co-polymer. The flexible ribbon may be optically clear in the range of about 400 nm to 700 nm, having a thickness in the range of about 0.04 mm to 1.0 mm, a width in the range of about 2.5 mm to 30 mm, and a length in the range of about 10 cm to 100,000 cm. However, it should be appreciated that the ribbon may have any other dimensions, as embodiments are not limited in this respect.

In one embodiment, the ribbon controller can be configured to guide the ribbon under the light receiver, e.g., at a substantially constant velocity.

In one embodiment, the light receiver can have a lens, e.g., a concave or convex lens, a plurality of lenses or any suitable type, and/or a magnifying lens. The light receiver can also include an image recording device, e.g., a still or video camera. The image recording device may be part of the light receiver or may be a separate device.

In one embodiment, the sample applicator can include an applicator pump.

In another embodiment the flexible ribbon can be replaced with a glass slide.

The system and method described herein can also include a diluent vessel in operable communication with the sample applicator. The diluent vessel may contain at least one diluent, and a diluent pump may be used to deliver one or more diluents from the diluent vessel to the sample applicator.

The system and method in accordance with some embodiments can also include a roll, cartridge or other component for dispensing the flexible ribbon, and a controller that is configured to receive the flexible ribbon from the roll or other dispensing component. The system can also include a collector to receive the pieces of ribbon containing stained cells following passage under the light receiver.

The system and methods can also include a computing device which may be any suitable computing device. The computer may comprise one or more processors and memory coupled with the processor(s). The memory may comprise one or more tangible, non-transitory, computer-readable storage media that may store computer-executable instructions. The computer-executable instructions, when executed by the processor(s), may cause the processor(s) to control operation of the described system. For example, the computer-executable instructions, when executed by the processor(s), may cause the processor(s) to instruct the sample applicator to dispense a sample onto the hydrophilic surface, instruct the diluent vessel to dilute or not dilute the sample, instruct the sample applicator to vary the distance between the tip and the flexible ribbon, instruct the ribbon controller to vary the distance between the ribbon and the sample applicator tip, instruct the ribbon controller to alter the tension of the ribbon, instruct the ribbon controller in what direction to guide the ribbon, instruct the ribbon controller to move the ribbon at a specified velocity, and/or change the velocity of the ribbon relative the light receiver, instruct the light receiver to adjust focus in response to a signal received by the light receiver, instruct the sample applicator to dispense a sample at a specific rate, instruct the sample applicator to dispense the sample for a specified time, or control any other operations of the components of the system.

Additional features and advantages of the invention will be made apparent from the following detailed description of illustrative embodiments which proceeds with reference to the accompanying figures.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic diagram of an exemplary system in which some embodiments may be implemented;

FIG. 2 is a schematic diagram illustrating an area where a cell monolayer is deposited, in accordance with some embodiments;

FIG. 3 is a schematic diagram illustrating exemplary processing of a sample, in accordance with some embodiments;

FIG. 4 is a schematic diagram illustrating a sample on a flexible ribbon being guided under a light receiver by ribbon controllers, in accordance with some embodiments;

FIG. 5 is a schematic diagram illustrating an enlarged view of some components of a ribbon controller, flexible ribbon, and sample, in accordance with some embodiments; and

FIG. 6A is a schematic diagram of an enlarged view of various shapes and geometries of sample applicator tips, in accordance with some embodiments;

FIG. 6B is another schematic diagram of an enlarged view of various shapes and geometries of sample applicator tips, in accordance with some embodiments;

FIG. 7 is a schematic diagram illustrating an embodiment in which the ribbon/glass slide controller includes a hub;

FIG. 8 is a schematic diagram illustrating an embodiment in which the sample applicator includes a capillary tube; and

FIG. 9 is a schematic diagram illustrating a sample applicator casting a monolayer of blood cells on a piece of hydrophilic polyester being pulled in a direction of an arrow shown, in accordance with some embodiments.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The applicant has recognized and appreciated that a flexible substrate may be used for preparing and handling biological and other types of samples for microscopic analysis. Accordingly, a flexible substrate, such as an optically clear, flexible ribbon may be provided that may be utilized in an automated microscopy system. At least a portion of the flexible ribbon can have a hydrophilic surface and can be used as a substrate for a monolayer of cells. The cell monolayer maybe continuous, which may significantly improve the quality of the sample analysis using an automated microscope.

Microscopic classification of blood cells and analysis of their morphology using existing approaches may have some drawbacks, which may be associated with preparation of a sample and subsequent steps. Generally, a blood smear needs to be created. The smear is fixed, stained, washed and then analyzed with a manual microscope. Such smears may then be assessed for the presence of a monolayer of cells. The monolayer portion of the smear is used to perform a five part differential measurement. If a monolayer is not created in the smear, the entire process of fixing, staining, and washing would need to be repeated.

Cellular enumeration with a microscope was generally performed with a hemacytometer so that cells and particles could be counted in a known volume of liquid. However, microscopes typically require the use of glass slides to perform a 5 part differential measurement. Glass slides can have some drawbacks in the automated analysis of cells. For example, wedge smears may be difficult to automate. Glass slides may require pre-treatment (corona discharge) to allow blood samples to be spread evenly on the surface of the glass slide. Moreover, glass slides may vary in thickness, requiring refocusing during microscopic examination. Processing a slide from a cell application to fixing, staining, washing, drying, and oiling typically requires complex mechanical automation that increases the complexity and cost of the instrument. In addition, glass slides may typically be unable to accommodate large volumes of sample. However, large volumes of sample may need to be analyzed when looking for rare cellular occurrences, e.g., metastatic cells in human blood, residual cancer cells after chemotherapy, or other artifacts.

The applicant has recognized and appreciated that use of a flexible ribbon can provide a number of advantages and can improve performance of microscopic and other techniques for analysis of samples. For example, the ribbon may allow a probe tip to have more positional flexibility relative to the surface of the ribbon, and by virtue of its hydrophilic surface may allow formation of a continuous cell monolayer. The monolayer can thus be formed precisely, with different blood samples yielding similar monolayers (including the widths of the monolayers).

The flexible ribbon can be cut into separate pieces, or portions, of any desired length. The separate portions of the ribbon may be transported for processing to respective different stations. This can allow simultaneous processing of different samples at a number of stations, which can improve efficiency, increase the throughput of the analysis, and decrease the amount of substrate consumed per sample.

As another advantage, because the described substrate, such as the ribbon having a sample deposited thereon, is flexible, it can be stretched, pulled or manipulated in any manner to position the sample with respect to a light receiving device (e.g., a microscope) in a desired orientation. Furthermore, the flexible ribbon may allow preparing a sample for analysis in a more efficient manner—for example, the sample can be stained, tagged and/or or otherwise prepared for microscopic or other type of analysis. In some cases, the sample can be thus prepared in advance, prior to depositing the sample onto the flexible substrate. The cell monolayer can have a desired width which may be selected based on a number of factors, such as a type of analysis, type of sample, and any other factors. In some embodiments, the width of the cell monolayer may be controlled such that a single pass or multiple passes (which may be done at different magnifications) through an optical axis of a microscope may encompass all or one or more portions of the cells disposed on the ribbon. In certain cases where the number of cells required to perform a test is small a single line of blood cells cast on a glass slide substrate may have certain advantages over a plastic material. These include ease of manual interrogation by a hematologist or pathologist and ease of labeling.

The substrate can be formed of a strong, pliable and flexible material, such as, for example, a polymer. The polymer may be a homopolymer, or copolymer, including alternating and block copolymers. Exemplary polymers used may be polyester (polyethylene terephthalate (PET)), polystyrene, and co-polymers thereof. The polymer can be a water insoluble polymer, and/or a non-water swellable polymer, as are known in the art or developed in the future.

The described techniques can be used in various clinical, medical, forensic, environmental and other applications. For example, the described techniques can be used in 5-part differential white blood cell analysis, a complete blood cell count, a CD4 T cell count, a reticulocyte count, detection of malarial parasites, detection of bacterial blood infections, and any other type of analyses.

In some embodiments, a system can be provided that can include suitable components configured to deposit a sample onto a flexible ribbon, preparing the sample for subsequent analysis and analyzing the sample. The sample can be deposited on at least a portion of the substrate in a form of a cell monolayer, which can be continuous.

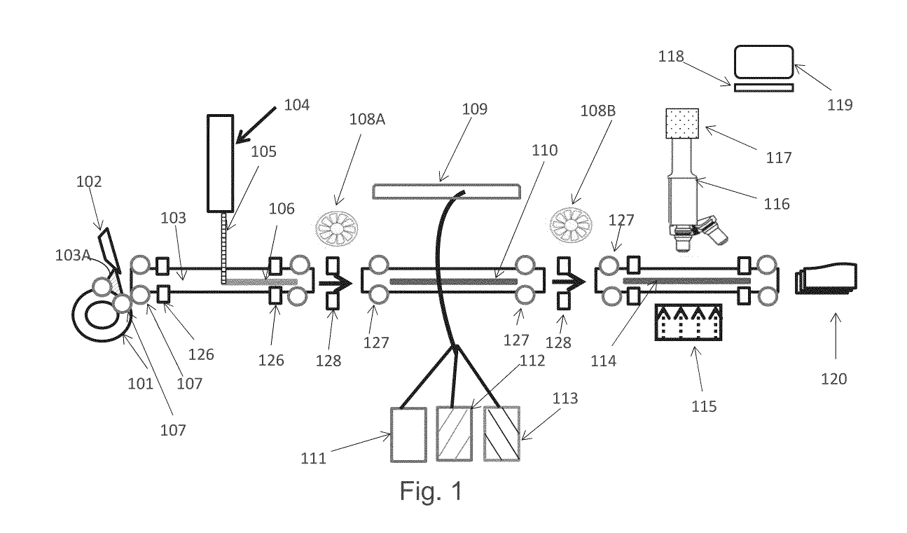

FIG. 1 illustrates one exemplary embodiment of a microscopy system. As shown, the system generally includes a flexible ribbon 103 that is stored on a roll 101, a ribbon controllers (e.g., rollers 107 and 127, and guides 126 and 128), a sample pump 104, a sample applicator 105, fans 108A and 108B, fluid controller 109 used to control the flow of fixing 111, staining 112, and washing 113 fluids, a light source 115, a light receiver 116, a camera 117, and a computing device 118 associated with a display device 119. The flexible ribbon 103 can have a hydrophilic surface. As shown in FIG. 1, ribbon 103 can be stored, and dispensed from a roll 101. The flexible ribbon 103 can be withdrawn by ribbon controllers (e.g., rollers 107) from roll 101. The ribbon controllers can guide ribbon 103 under the tip end of sample applicator 105. Ribbon controllers, such as guides 126 and 128, can position ribbon 103 at a fixed distance from the tip end of sample applicator 105. As ribbon 103 is advanced along the system with rollers 107 and guides 126, a sample pump 104 causes sample 106 to be dispensed from the tip end of the sample applicator 105 on to a hydrophilic surface of the flexible ribbon 103. Fan 108A, or any other apparatus, can be used to dry the sample as the cell monolayer is formed or after the formation of the monolayer. Ribbon controllers (e.g., rollers 127 and guides 128) continue to advance the ribbon 103 to position the ribbon 103 under the fluid controller. The fluid controller 109 can seal around the monolayer segment 110, and can sequentially add and then aspirate off (after certain time periods) fixing fluid 111, staining fluid 112, and washing fluid 113. Fan 108B can be used to dry the stained cell monolayer 110. Ribbon controllers (e.g., rollers 127 and guides 128) continue to advance the ribbon 103 to position the ribbon 103 under a light receiver 116. A light source 115 can be provided to allow a light receiver 116 to receive light, which is used to receive information about the sample.

A suitable computing device may be used to store and process any data acquired using the sample analysis. The light-receiving device, such as a microscope, can be equipped with camera 117, which can be an integral part of the device or can otherwise be associated with the microscope. Camera 117 may comprise a charge-coupled device (CCD) or any other image acquisition device. Following collection of data, ribbon 103 may be discarded, stored for further analysis, or otherwise manipulated.

Cutter 102 can be provided to cut ribbon 103 to one or more pieces of a suitable length. For example, FIG. 1 illustrates that ribbon 103 may be cut and a ribbon portion 103A may be left as the tail to roll 101, e.g., to be grasped by ribbon controllers for processing of the next sample. It should be appreciated that the microscopy system can include any other suitable components that are not shown herein for the sake of simplicity of representation.

In some embodiments, a computing device (e.g., computing device 118) comprising one or more processor(s), memory coupled with the processor(s), and any other suitable components can be utilized to control one or more components of the microscopy system. Computing device 118 can include any other suitable components and devices. As shown in FIG. 1, computing device 118 may be associated with display 119 which may be a separate device or may be an integral part of computing device 118 (e.g., when computing device is a tablet, laptop, smartphone, PDA, or other device).

The memory of the computing device may comprise one or more tangible, non-transitory, computer-readable storage media that may store computer-executable instructions. Non-transitory computer-readable storage media may include but are not limited to magnetic storage devices (e.g., a hard disk, floppy disk, and magnetic strips, among others), optical disks (e.g., a compact disk (CD), digital versatile disk (DVD), and other media), smart cards, and flash memory devices (e.g., card, stick, and key drive, among others). In contrast, computer-readable media generally (i.e., not necessarily storage media) can additionally include communication media such as transmission media for wireless signals and the like. The computer-readable storage having the computer-executable instructions may also be referred to as “software” or “computer software.”

The computer-executable instructions, when executed by the processor(s), can cause the processor(s) to control operation of one or more components of the described system. For example, the computer-executable instructions, when executed by the processor(s), can cause the processor(s) to provide instructions to ribbon controllers. For example, rollers 107 can be controlled to indicate a direction to move ribbon 103, a velocity to the movement of the ribbon, amount of tension to apply to the ribbon, and any other suitable parameters. Guides 126 can be controlled to change their position such that to increase or decrease the distance between the ribbon and the sample applicator tip end, change the position of guides 126 to increase or decrease the distance between the ribbon and the light receiver, change the position of guides 126 to increase or decrease the distance between the ribbon and light source, and/or adjust the tension on the ribbon.

The computer-executable instructions, when executed by the processor(s), can cause the processor(s) to provide instructions to sample applicator 105. Such instructions can include whether to dispense a sample, volume of sample to dispense, and/or whether to dilute the sample. The computer-executable instructions, when executed, can also be used to control one or more light sources utilized in the described system. For example, the computer software can be used to control whether a filter is used in association with the light source, select a particular light source or type of light source, and/or set and adjust any suitable parameters of the light source or other components of the system.

The computer software, when executed, can provide instructions to light receiver 116 and camera 117 to control operation of the devices in any suitable way. Such instructions can include, for example, altering the distance between the light receiver and the ribbon. In embodiments where the light receiver includes a microscope, such instructions can include adjusting the focus motor, or magnification, or controlling any other aspects of operation of the microscope. It should be appreciated that embodiments are not limited with respect to controlling component(s) and device(s) of the described system using the computing device, and any type of operation can be controlled in a suitable manner.

Additionally or alternatively, the computing device can receive user instructions to control operation of one or more components of the described system. A user can be enabled to set and adjust one or more parameters of the components in any suitable manner, for example, via a user interface of display 119.

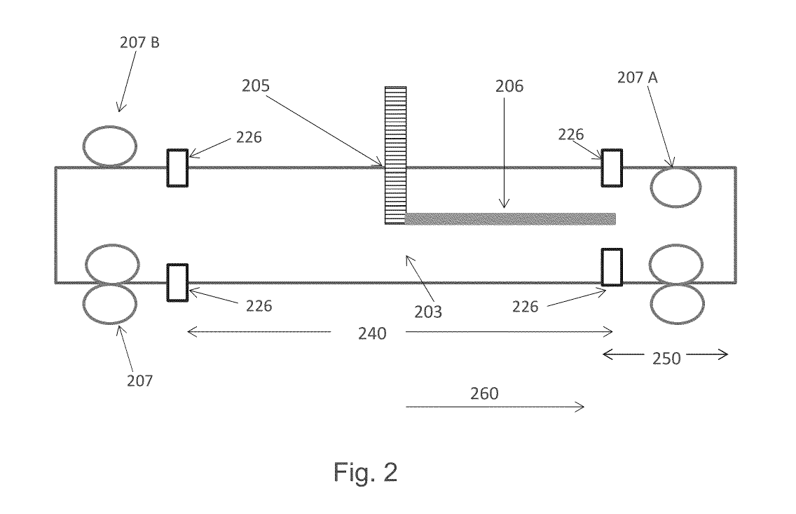

FIG. 2 illustrates exemplary components of a device or system which controls a substrate, such as a flexible ribbon, in accordance with some embodiments. The device which operates to move the ribbon is referred to herein as a ribbon controller by way of example only. As shown in FIG. 2, flexible ribbon 203 (which may be similar to ribbon 103 shown in FIG. 1) having a hydrophilic surface can be disposed under a sample applicator tip 205. Ribbon controller rollers can be positioned differently with respect to ribbon 203. For example, as schematically shown in FIG. 2, ribbon controller rollers can be positioned above (207A), below (207B), or above and below (207) ribbon 203.

In some embodiments, a stepper motor or other actuator (not shown) can be used to drive the rollers or other components for moving the substrate. The motor and other components can be controlled by a computing device comprising one or more processors that execute computer-executable instructions stored in memory of the computing device. The motor may control the speed of the rollers. The motor can be controlled to operate in any suitable direction—e.g., in a forward or reverse direction.

In some embodiments, the ribbon may be tensioned by sets of guides and/or rollers. In one embodiment, the tension roller(s) may also be driven by a stepper motor. The motors can also reverse direction so that tension rollers operate as the drive rollers, and the drive rollers operate as the tension rollers.

Guides 226 can be utilized to center the hydrophilic surface of the substrate under the sample applicator. For example, guides 226 can also be utilized to stretch ribbon 203 to create a length of ribbon 203 which can thus be under tension in an area schematically indicated as 240 in FIG. 2. Guides 226 can also be utilized to center the ribbon along a light receiver having an optical axis, as well as move ribbon 203 closer or further from the sample applicator tip. In some embodiments, the ribbon can be guided under the light receiver while the ribbon is under tension. Additionally or alternatively, an area of ribbon 203 which is under no tension (250 in FIG. 2) can be guided under the light receiver. In the example shown in FIG. 2, ribbon 203 can be moved in the direction of arrow 260. Though, it should be appreciated that ribbon 203 can be moved in the opposite direction as well.

As shown in FIG. 2, a biological sample 206 (e.g., whole blood) may be deposited on flexible ribbon 203 in a single column. The tip end of the sample applicator 205 is positioned above the hydrophilic surface of ribbon 203. The tip end can be then brought into close proximity with the hydrophilic surface of ribbon 203. The ribbon, driven by the rollers, can start moving at a rate of about 20-30 mm/sec. The sample applicator can operate to dispense the sample onto the ribbon at a rate of 0.05 μL/sec for 5-6 seconds. By varying the size of the tip opening, the velocity of the ribbon, and volume of the dispensed sample, various widths of sample can be dispensed. For example, in some embodiments, the column can have a width of about 0.6 mm to about 2.0 mm. Though, it should be appreciated that a column of any suitable width can be formed, as embodiments are not limited in this respect.

It should be appreciated that ribbon 103 (FIG. 1) and ribbon 203 (FIG. 2) may comprise pieces of ribbon. In such embodiments, ribbon controllers can continuously advance each flexible piece of ribbon to the staining station while different samples are simultaneously applied onto the hydrophilic surface of a different piece of ribbon and a third piece of ribbon brought under the light receiver by ribbon controllers. Alternatively, in embodiments where a large volume of sample is required for analysis, the same sample can be applied continuously to the flexible ribbon as the flexible ribbon is brought under the light receiver by the ribbon controllers.

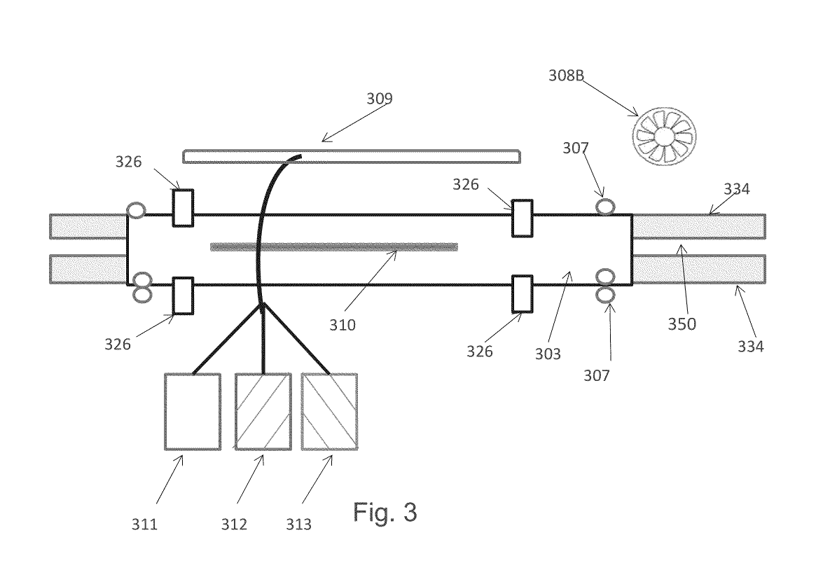

FIG. 3 illustrates schematically that a sample 310 on a flexible ribbon 303 (which may be similar to ribbon 103 in FIG. 1 and/or ribbon 203 in FIG. 2) can be advanced under fluid controller 309. Rollers 307 can be positioned above and below flexible ribbon 303. Rollers 307 can be controlled with motors, and the motors and rollers can reverse direction, moving the ribbon in a forward or reverse direction. Guides 326 can be utilized to align ribbon 303 and sample 310 with fluid controller 309. The fluid controller 309 can come down and seal against ribbon 303 by pressing a gasket positioned around the fluid controller head against the ribbon which is supported by two flat aluminum plates 334. The fluids (fixing solution 311, staining solution 312, and washing solution 313) can be moved onto ribbon 303 sequentially using peristaltic pumps (not shown), and removed from 303 by a vacuum pump (also not shown). It should be appreciated, however, that the fluids 311, 312, and 313 may be applied onto ribbon 303 in any suitable manner.

The monolayer sample 310 can be fixed by pumping the fixing solution 311 onto the sample 310 and flexible ribbon 303. After a certain period of time (e.g., 30 seconds in one embodiment) the fixative 311 is aspirated off, and staining solution 312 is pumped in. The staining solution 312 may be allowed to incubate (e.g., from about 1 to 2 minutes), and is aspirated off by vacuum. The washing solution 313 can then be pumped onto sample 310 under the fluid controller head. It can be incubated for a certain period of time (e.g., 30 seconds) and is aspirated off. The fluid controller head 309 is raised up and off ribbon 303. Rollers 307 and guides 326 may then move the sample 310 to a position such that ribbon 303 is dried off, for example, by fan 308B. It should be appreciated that the above steps of applying fixing, staining and washing solutions to the sample are shown by way of example only. The sample deposited onto a substrate in accordance with some embodiments may be prepared in any suitable manner as embodiments are not limited in this respect.

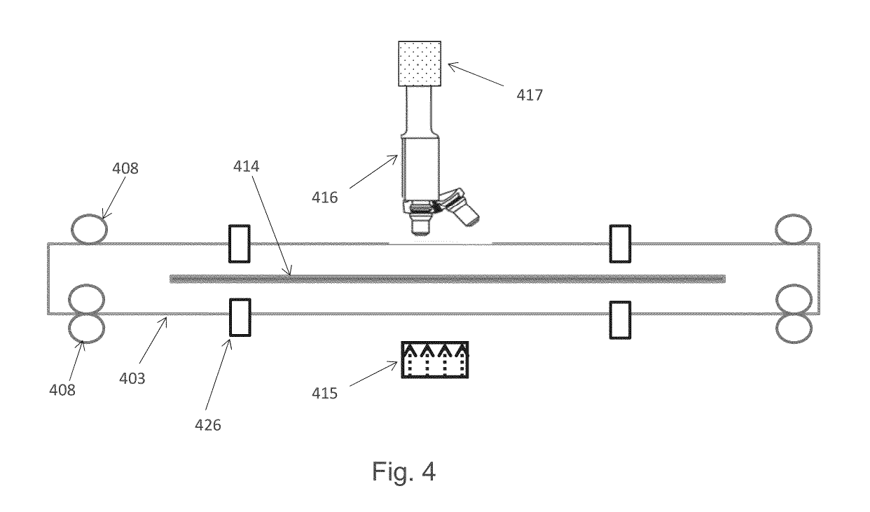

FIG. 4 illustrates schematically that a sample 414 on a flexible ribbon 403 (which may be similar to ribbon 103 in FIG. 1) can be advanced under a light receiver 416, such as a microscope. Rollers 408 can be positioned above and below flexible ribbon 403. Rollers 408 can be controlled with motors, and the motors and rollers can reverse direction, moving the ribbon in a forward or reverse direction. Guides 426 can be utilized to align ribbon 403 and sample 414 with light receiver 416. As shown in FIG. 4, guides 426 can align the sample deposited on ribbon 403 along the optical axis of a light receiver 416. Light receiver 416 can be associated with a light source 415, camera 417 that can record images of the sample, and any other suitable components. A suitable computing device which can be communicatively coupled with light receiver 416 can be used to process and analyze images acquired by camera 417.

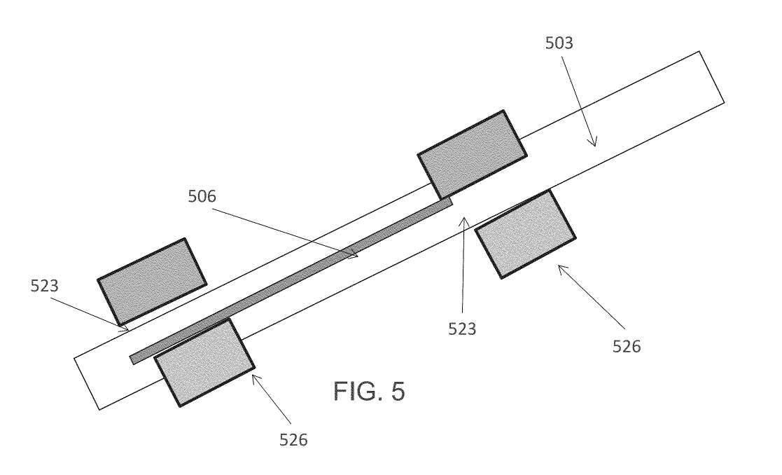

FIG. 5 illustrates an embodiment in which ribbon controller guides 526 (only the top part of guide is shown by way of example) may span the width of a ribbon 503. Each of guides 526 must have a slot 523 which allows for passage of the sample deposited on the hydrophilic surface (not shown) of the flexible ribbon. Ribbon 503 can be guided under tension, e.g., stretched by slightly tilting the guides down, to prevent damage to the column of sample 514, e.g., blood elements. This arrangement allows the monolayer to remain in focus with only minor adjustments as the blood elements are drawn through the optical axis of a light receiver, such as a microscope.

Substrate

The substrate utilized in some embodiments can be an optically clear, thin ribbon, formed of a strong, pliable and flexible material, such as, for example, a polymer. The polymer can be a homopolymer, or copolymer, including alternating and block copolymers. Exemplary polymers used can be polyester (polyethylene terephthalate (PET)), polystyrene, and co-polymers thereof. The polymer can be a water insoluble polymer, and/or a non-water swellable polymer, as are known in the art or developed in the future.

In one embodiment, at least a portion of a surface of the ribbon can be a hydrophilic surface. In one embodiment, only one portion of one side of the flexible ribbon has a hydrophilic surface. In another embodiment, the entire side of one side of the flexible ribbon has a hydrophilic surface. In another embodiment, only one portion of both sides of the ribbon has a hydrophilic surface. In another embodiment, the entire surfaces of both sides of the ribbon have a hydrophilic surface.

Without intending to be bound by theory, it is believed the hydrophilic surface of the ribbon allows for aqueous samples to be spread in thin layers, i.e., resulting in the creation of a monolayer of cells. Aqueous solutions generally have a high surface tension, causing them to “bead” and “withdraw” on substrates generally used in light microscopy techniques, i.e., glass and polymers having hydrophobic surfaces. The combined high surface tension of the sample and hydrophobic nature of the substrate generally can prevent the formation of a monolayer of cells in the absence of use of cover slides or other surface tension reducing agents. However, the use of cover slides can be labor and cost intensive. Addition of surface tension reducing agents (i.e., surfactants) to aqueous samples can cause a change in the nature of the sample, i.e., adversely affecting morphology of cells. Furthermore, use of hydrophilic polymers as microscopy slides can be difficult. Hydrophilic polymers typically lack the transparency and clarity required for light microscopy, which is traditionally provided by use of glass and hydrophobic polymers. Furthermore, hydrophilic polymers generally swell, and/or degrade when aqueous solutions are applied thereon. Without intending to be bound by theory, it is believed the hydrophilic surface of the ribbon utilized in some embodiments provides greater molecular interaction with the aqueous sample, allowing for the formation of a thin film, i.e., a monolayer of cells. In one embodiment, the hydrophilic surface is not water soluble, and/or does not alter, increase, or decrease the osmolality of the sample being applied. In another embodiment, the hydrophilic surface of the flexible ribbon is a solid surface, and the hydrophilic surface is not a liquid surface.

In addition to the flexibility offered by the described system, a number of other advantages can be realized by using the flexible ribbons having a hydrophilic surface. In contrast to rigid slides, the flexible ribbon does not require an exact close positioning of the applicator tip, e.g., a needle or tube, to the surface of the slide. Furthermore, the capillary or blunt tipped needle can be positioned more flexibly close to or just touching the flexible ribbon without damaging or tearing the ribbon, and/or affecting the creation of a monolayer of blood elements. Furthermore, a computing device can be used to control the formation of a monolayer of cells with factors other than simply altering the distance between the sample applicator tip and the ribbon.

The term “optically clear,” as used herein, refers to the fraction of light at specific or various wavelengths that pass through the ribbon, or is reflected from the ribbon. Light can include visible light, as well as light of other wavelengths in the electromagnetic spectrum, e.g., radio waves, microwaves, infrared radiation, ultraviolet radiation, X-rays and gamma rays. In some embodiments, the light comprises electromagnetic radiation in the visible spectrum, i.e., having a wavelength from about 400 nm to about 700 nm.

In some embodiments, a light source is provided, which provides illumination of the ribbon and hydrophilic surface as either a transmitted ray, or a reflected ray. In cases where the ribbon is illuminated with transmitted rays, optically clear means at least 85% of the incident ray passes through the ribbon to the light receiver. In one embodiment, the ribbon can have a transmittance of at least 85% between about 400 nm and about 700 nm.

In another embodiment, optically clear means at least 85% of the incident ray pass through the ribbon and hydrophilic surface. In one embodiment, the ribbon and hydrophilic surface have a transmittance of at least 85% or more for light between about 400 nm and about 700 nm. In some embodiments, the ribbon is essentially transparent to light down to a wavelength of 400 nm.

Whether or not a flexible ribbon and/or a hydrophilic surface are “optically clear” can be determined using any suitable techniques as are known in the art by one of skill in the art, for example, by use of a spectrophotometer, or other similar analytical light measuring device.

For ease of use, the ribbon can be cut and/or stored as pieces or forms of suitable size and shape, and packed into a suitable container. The ribbon can be packaged as a roll, or cassette including hundreds of meters of material in a small package. The ribbon can be cut into segments with a cutter, processed, labeled and saved, e.g., in a collection rack.

The substrate in accordance with some embodiments can comprise a flexible ribbon which can be of any suitable size. The ribbon can have a length, width and thickness. The width of the ribbon can be selected based on various factors, such as a type of analysis to be performed on the sample being analyzed, characteristics of the sample (e.g., a type of the sample, a volume of the sample, and any other suitable characteristics), a velocity of the ribbon that can be guided by the ribbon controller, and any other suitable factors. In some embodiments, the width of the ribbon can be greater than approximately 2.5 mm, and less than approximately 25 millimeters.

In one embodiment, the entire ribbon or any portion of the ribbon (e.g., the hydrophilic surface) can be formed of a material which is compatible with solvents used in analysis of blood samples. Any suitable solvents as known in the art can be used. For example, polyethylene terephthalate (PET) is resistant to solvents, such as methanol, and dyes. Accordingly, PET can be used with fixatives, stains, dyes, and wash solutions, as known in the art.

The ribbon utilized in some embodiments is pliable and flexible. Accordingly, the ribbon stored on a spool, and when unrolled can remain flat. The ribbon can also be stretched under ribbon controllers, e.g., parallel guides, to receive a sample from a sample applicator, and also under other ribbon controllers to align the ribbon during microscopic analysis, i.e., through the optical axis of the microscope. These features allow for the continuous dispensing and analysis of blood elements. For example, up to 500 meters of the ribbon could be used for dispensing blood elements. The volume of blood or cell growth media dispensed over this distance is far greater than can be dispensed on hundreds of rigid slides. The flexible ribbons can also include other elements useful in the analysis of samples. For example, calibration indicia can be printed on the flexible ribbon or the hydrophilic surface. Images of the calibration indicia received by the light receiver can be utilized by the computer to continuously focus the image by utilizing ribbon controllers to move the ribbon along the optical axis of the light receiver, adjust the focus of the light receiver, or both. Images of unique calibration indicia can also be recorded by the camera so that cells of interest can easily be located on the length of the flexible ribbon if the ribbon is selected for further analysis by a person.

Ribbon Controller

The flexible ribbon in accordance with some embodiments can be guided and advanced with one or more types of ribbon controllers. Various types of ribbon controllers can be used.

Rollers can be utilized to move a ribbon in a horizontal axis, e.g., along a path from the sample applicator to the light receiver, and are configured to receive the flexible ribbon. Motors known to those of skill in the art can be used to control the speed of the rollers, e.g., via instructions from a computer. Motors are able to reverse direction, and thus, roller and direction of movement of the ribbon can also be reversed. Rollers can be used to move the ribbon at a controlled, constant, and/or variable velocity of between approximately 1 mm/second and approximately 50 mm/second. At certain points, it can be desirable to maintain the rollers at a zero velocity, for example, when the sample is present at the staining area.

Guides are utilized to center the flexible ribbon along a vertical axis, e.g., along the optical axis of a light receiver, or vary the distance between the hydrophilic surface and sample applicator. The guides can be operated by a motor. Rollers and guides can act in concert to maintain a length of ribbon under tension.

In some embodiments, supports can be used in conjunction with rollers and guides. Generally, a support provides sufficient rigidity to prevent bending of the flexible ribbon. In such embodiments, the ribbon and support are thus controlled by guides and rollers, e.g., in a horizontal or vertical direction. In one embodiment, the support comprises two parallel rigid edges which form an aperture there between, e.g., along the length of the flexible ribbon. The hydrophilic surface of the ribbon is congruent with the aperture, allowing for the sample applicator to depress the flexible ribbon as a sample is dispensed therefrom. Furthermore, the aperture allows for light to pass through the sample and hydrophilic surface, e.g., to the light receiver.

Sample Applicator

A sample applicator in accordance with some embodiments can dispense a sample to the hydrophilic surface of the flexible ribbon either as a spot, a column, or in any other suitable way. The sample can be a biological sample, e.g., blood. The sample is dispensed on to the hydrophilic surface through the tip end of the sample applicator, forming a monolayer of cells.

The sample applicator can include a pump as is known in the art, e.g., a positive displacement pump which can be a piston pump or a syringe pump. The displacement pump can be connected through a tube to a blunt tipped stainless steel needle, which forms the tip end. The tip of the sample applicator can be made of any material known to those of skill in the art, including stainless steel, glass or plastic.

For blood samples, the aspiration rate for the pump can be between 0.02 ml/sec and 0.5 ml/sec, and, in some embodiments, 0.05 ml/sec and 0.25 ml/sec. The dispense rate for the pump can be between 0.01 μl/sec and 0.4 μl/sec, and, in some embodiments, 0.02 μl/sec and 0.2 μl/sec. The volume of sample dispensed on the hydrophilic surface of flexible ribbon can vary from less than a 1 microliter to more than 5 ml.

In one embodiment, the sample applicator and/or tip can include a capillary tube which can be replaceable and/or disposable. The capillary tube can be used to collect and process cells drawn into the capillary tube. The capillary tube can be manually or automatically inserted into the sample applicator before the monolayer is cast. The capillary tube can have an end for aspirating the blood sample, and a tip end for casting the monolayer.

In some embodiments, the tip of the sample applicator is a fixed part of the sample applicator, and can be washed in between dispensing samples. In other embodiments, the tip of the sample applicator can be replaceable and/or disposable, and a new tip can be used as each new sample is processed.

The inner diameter of the sample applicator or a tip of the sample applicator can determine the width of the cell monolayer cast. The width of the cell monolayer can also be determined and/or established by the speed of the ribbon moving under the sample applicator, and the rate the sample is pumped out of the applicator. Unlike solid substrates, a flexible ribbon 103 allows for more flexible positioning with the tip 105 without significantly affecting the formation of the monolayer or the width of the monolayer. Both of these characteristics are important in creating a cell monolayer using a capillary tube as a tip.

In some embodiments, the blood drawing end of a capillary tube has an internal diameter of between about 0.4-3.0 mm, preferably 0.8-1.5 mm and an outside diameter of between about 0.6-5.0 mm, preferably 1.2-2.5 mm. In other embodiments, the blood drawing end of a capillary tube has an internal diameter of between about 0.5-2.6 mm, preferably 1.0-1.3 mm and an outside diameter of between about 0.7-4.4 mm, preferably 1.5-2.2 mm.

In some embodiments, the capillary tube dispensing tip end has an inner diameter of between about 0.2-2.0 mm, preferably 0.4 to 1.0 mm and an outer diameter of between about 0.3-2.4 mm, preferably 0.6-1.2 mm. In other embodiments, the capillary tube dispensing tip end has an inner diameter of between about 0.3-1.6 mm, preferably 0.6-0.8 mm and an outer diameter of between about 0.5-2.0 mm, preferably 0.8-1.0 mm.

In some embodiments, the capillary tube can be asymmetric in size, having a smaller internal diameter and a smaller external diameter on one end to provide optimal blood element dispensing properties, and a larger internal diameter and larger external diameter to provide optimal capillary action.

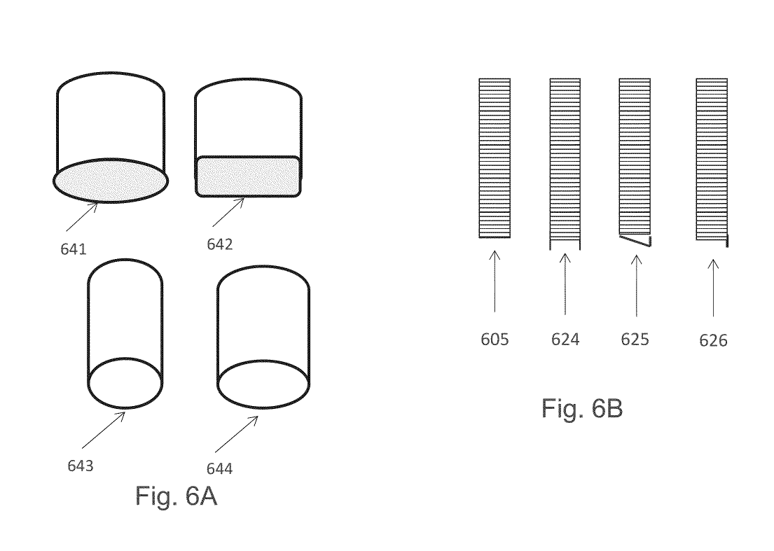

The tip of the sample applicator can have various shapes, sizes, and geometries. Referring to FIG. 6A, the tip end can have various cross sections and sizes, including, for example, an elliptical cross section 641, substantially rectangular cross section 642, circular cross section 643, or elliptical cross section 644. The tip can also have different shaped end profiles, such as flat 605, groove 624, angled 625, or irregular 626, as shown in FIG. 6B. Different geometric designs and profiles of the tip of the sample applicator can facilitate application of the sample, depending on the composition of the sample, e.g., formation of a monolayer of cells.

Prior to application onto the hydrophilic surface of the flexible ribbon, cells from a sample can be processed within the disposable sample applicator (capillary tube) via various methods and materials known to those of skill in the art. Thus, in one embodiment, the sample applicator can contain materials such as dyes and anti-coagulants. For example, the disposable sample applicator can be coated with heparin or EDTA in an amount to prevent blood clotting in the sample applicator. The sample applicator can also comprise other agents, including dyes, such as, for example, methylene blue and/or eosin. Other coatings for the inside of the tip can include molecular probes to identify blood elements of interest to clinicians such as CD4 T cells. Such molecular probes can include antibodies, e.g., fluorescent labeled antibodies. In other embodiments, the molecular probe is an antibody directed against CD4 cells with a fluorescent marker linked to the antibody. In some embodiments, probes are directed against cell surface receptors. In other embodiments, molecular probes are provided which are directed against peptides, proteins, nucleic acids, bacteria, and/or viruses.

It is contemplated that the volume of liquid cast onto the ribbon can be selected and/or changed by an operator, or automatically selected and/or changed via computer control. In one embodiment, a sample can be applied quantitatively so that the observed images can be used to perform tests such as, for example, complete blood count, CD4 T cell enumeration, extent of malarial infection, and apoptosis assessment.

Diluent Vessel

In one embodiment, a diluent vessel is provided which is in operable communication with the sample applicator. Depending on the sample being processed, it can be desirable to dilute the sample. The diluent vessel can comprise at least one compartment including at least one diluent. The diluent vessel can also include a diluent pump to deliver the diluent to the sample applicator. A computing device can be used to instruct the diluent vessel to deliver a volume of diluent to the sample applicator. Any suitable diluents can be utilized, which can be selected based on a type of the sample being dispensed from the sample applicator and/or any other suitable factors.

Light Receiver

Light receivers are known to those of skill in the art, and are generally devices for receiving an image of the sample, e.g., a bright field microscope. Light receivers can have a lens and a focus motor to center and/or focus the image of the sample along an optical axis of the lens. The light receiver can include image receiving and recording devices, which can be a still camera, video camera, a line scanning camera, or any other optical device. In one embodiment, a cell monolayer on the ribbon can be automatically centered by ribbon controllers along the optical axis of the light receiver, e.g., to produce a clear, non-blurry image.

Depending on the particular application, one of skill in the art can select an appropriate light receiver. In some cases, the light receiver can include a camera and microscope to receive light transmitted through the flexible ribbon. In other cases, fluorescent emissions from cellular and non-cellular objects can be detected by the light receiver.

In some embodiments, more than one light receiver can be utilized. For example, a first light receiver can permit faster data accumulation from the entire width of the cell monolayer using a low magnification. Additionally or alternatively, the first light receiver can detect fluorescence associated with different abnormal rare cells, and record the location of those cells. A second light receiver can require the ribbon to move at variable speeds, and ribbon controllers can position the ribbon such that the abnormal, rare cells are under the second light receiver. The second light receiver can present a high magnification bright-field and fluorescent image with the field of view spanning only part of the width of the cell monolayer.

In some embodiments, the light receiver can be able to analyze most of the sample deposited on the hydrophilic surface of the flexible ribbon. Preferably, the sample is deposited as a column having a width, such as in a form of a cell monolayer. The width of the monolayer of cells dispensed on the hydrophilic surface of the flexible ribbon can be controlled, e.g., by controlling the sample applicator and/or ribbon controller. Thus, a single pass or multiple passes (at different magnifications) the light receiver can analyze all cells in the monolayer, or at least a certain percentage of the cells placed upon the ribbon (e.g., at least 20-30%).

Light Source

A light source utilized in some embodiments can be a suitable light source that can be used for light microscopy. For example, a flash lamp, arc lamp, or halogen lamp can be used for generating white light. A rotational motor or other suitable device can be utilized for bringing filters of different wavelengths into the path of the light. In other embodiments, a plurality of LED's can be used as the light source. For example, a first LED is provided to emit light at a first wavelength and a second LED is provided to emit light at a second wavelength. As a non-limiting example, a first wavelength can be about 570 nm and a second wavelength can be about 430 nm. The LED's can operate independently, or together. It should be appreciated any suitable number of LEDs or light sources of any other type can be utilized.

In embodiments where fluorescent markers are detected in the sample, the light source may be positioned on the same side of the ribbon as the light receiver, and the light source can be coupled into the optical axis by using a dichroic beam splitter. In such embodiments, spectral filters, termed excitation filters and emission filters as are known in the art, may be used to substantially reduce the amount of light from the light source that may enter the light receiver, and allow substantially only fluorescently emitted light from the sample to enter the light receiver. This arrangement is typically known in the art of microscopy as epifluorescence illumination.

In some embodiments, the image can be refined by compensating for spatial shifts and distortions caused by movement of the ribbon. In another embodiment, images can be derived from two or more separate wavelengths of light, and the two or more images received by the light receiver can be combined together by suitable computer software executed by a computer to create a multi-color image.

Computing Device

In some embodiments, one or more computing devices that can be used to process data acquired using the described techniques. The computing device can be a PC, laptop, smartphone, tablet computer, PDA, or other computing device. The computing device can comprise at least one processor and memory coupled with the processor(s). It should be appreciated that the computing device can comprise any other suitable components.

The memory can comprise at least one tangible, non-transitory, computer-readable storage media that can store computer-executable instructions. The non-transitory computer-readable storage media can include but are not limited to magnetic storage devices (e.g., hard disk, floppy disk, and magnetic strips, and any others), optical disks (e.g., compact disk (CD), and digital versatile disk (DVD), among others), smart cards, and flash memory devices (e.g., card, stick, and key drive, among others). In contrast, computer-readable media generally (i.e., not necessarily storage media) can additionally include communication media such as transmission media for wireless signals and the like.

The non-transitory computer-readable storage media can store computer-executable instructions (e.g., computer software) that, when executed by the processor(s) cause the computing device to control operation of the light receiver, sample applicator, ribbon controller, and any other components that can be utilized to implement the described techniques. The computing device can be communicatively coupled with any of these components and can transmit signals to the components and receive information from the components. For example, in some embodiments, the computing device can receive an indication from one or more of the components to instruct, for example, the sample applicator or ribbon controller to modulate the thickness of the cell layer dispensed onto the ribbon. The software can also allow the system to analyze and categorize the cell and particle images captured by the camera. The computer software, when executed, can be used to control operation of various components to manipulate the sample in the sample applicator in a suitable manner. For example, when a capillary tube is used, the computer software can instruct the sample applicator to move the blood up and down in the capillary tube to mix the dyes and fluorescent tags with the blood. Through an analysis of the data the computer software can calculate the number of each cell type and particle identified, and the distribution of sizes. Abnormal morphological images can be isolated and stored for review by a user. The computer software can also be used to selected images to be stored to future review by a user.

Gas Movement

In certain embodiments, movement of air across the surface of the ribbon can be desirable, e.g., with a fan, bellows, or other similar devices. Such air movement can be dependent on the sample being analyzed, and the particular assay being performed, e.g., whether or not cells need to be dried, fixed, and/or dried after being fixed, stained, and washed.

Although the systems and methods in accordance with some embodiments are described herein as used for the analysis of blood, it is contemplated that other liquid biological samples can also be analyzed with appropriate pre-treatment, such as dilution with a diluent. Such samples can include, for example, bone marrow, amniotic fluid, breast milk, cerebrospinal fluid, chyle, exudates, lymph, mucus, pericardial fluid, peritoneal fluid, pleural fluid, pus, saliva, semen, synovial fluid, tears, and urine. Furthermore, samples that can be analyzed using the described techniques can comprise clinical samples or any other types of samples—for example, tissue culture samples, bacterial cultures, environmental samples (e.g., water and any other liquids), food samples, and other materials which can be subject to quality control inspection.

The described techniques can be useful in screening methods, e.g., drug discovery. As the described techniques can be used to rapidly and automatically screen samples, the characteristics of a vast number of compounds can be quickly examined, e.g., in their ability to enhance or inhibit the growth of microorganisms or cancer cells, and in a large number of other applications.

The described techniques can be useful in diagnosis of diseases, or conditions. Furthermore, the described techniques can be useful in the treatment, prevention, or reducing the risk incidence of suffering from such diseases or conditions, e.g., by early detection.

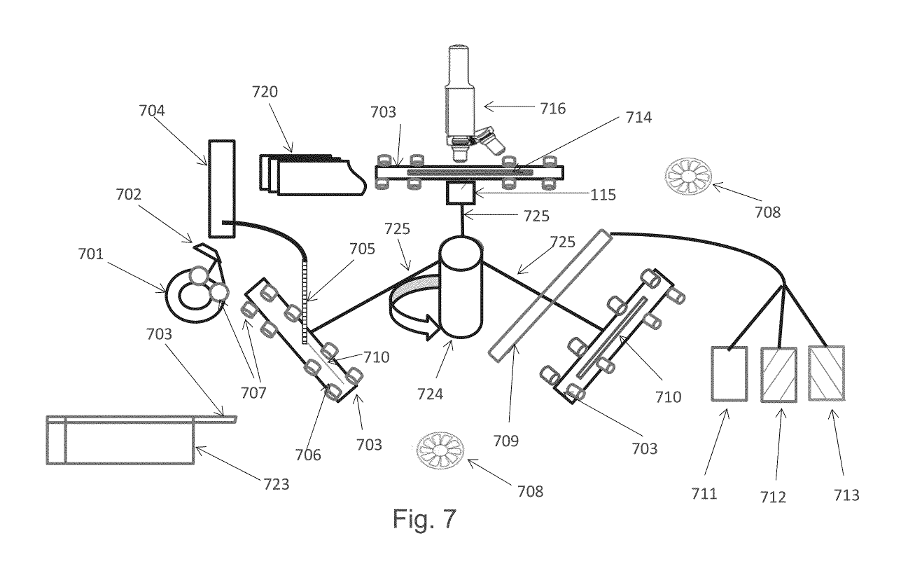

FIG. 7 illustrates exemplary components that can be used to implement the described techniques and can be similar to components shown in FIG. 1. However, a microscopy system as shown in FIG. 7 can be arranged in the form of a motorized turntable or hub, wherein a motorized arm forms a part of a ribbon controller. A ribbon 703 can be stored as roll 701 or cassette 723. Ribbon controller 707 grips and advances ribbon 703 under sample applicator 704 and tip 705, wherein sample 706 is applied to the hydrophilic surface of the flexible ribbon 703. Ribbon 703 can be cut with cutter 702, and the ribbon is maintained under tension by ribbon controller 707. In one embodiment, ribbon 703 is maintained on top of a support (not shown), which is held by arm 725. Arm 725 is mounted to a central revolving hub 724, which then revolves and passes ribbon 703 in front of fan 708 to be dried. The revolving hub then moves the ribbon to a sample processing area, where stains can be applied to a sample 710. Following treatment of sample 710 with fixative 711, stain 712, and wash 713 under fluid dispensing head 709, hub 724 moves ribbon 703 to light receiver 716. At the light receiver, light source 715 is provided, and images of the sample 714 can be captured with a camera (not shown). In this embodiment, the three processes can be performed simultaneously on three different lengths of the flexible ribbon, and the ribbon can be collected following analysis, for example, for storage and/or further analysis.

Cassette 723 can also be used to hold glass slides. The slide controller 707 grips and advances the slide 703 under the sample applicator 704 and tip 705, where in sample 706 is applied to the hydrophilic surface of the glass slide 703. Arm 725 mounted to a central revolving hub 724, which then revolves and passes slide 703 in front of fan 708 to be dried. The revolving hub then moves the slide to a sample processing area where stains can be applied to sample 710. Following treatment of the sample 710 with fixative 711, stain 712, and wash 713 under fluid dispensing head 709, hub 724 moves slide 703 to light receiver 716. At the light receiver, light source 715 is provided, and images of the sample 714 can be captured with a camera (not shown). In this embodiment the three processes can be performed simultaneously on three different slides, and the slides are then labeled and collected for storage and/or further analysis.

As shown in FIG. 7, arms 725 can be utilized to move the flexible ribbon around hub 724. It is contemplated that other structures and mechanical devices can be utilized as a ribbon or glass slide controller. In other embodiments, the ribbon controller can include a disk. Thus, the arms of FIG. 7 can be replaced with one or more disks, so that flexible ribbon 703 is held in place against the disk with ribbon controllers, and the disk rotates with the revolving hub.

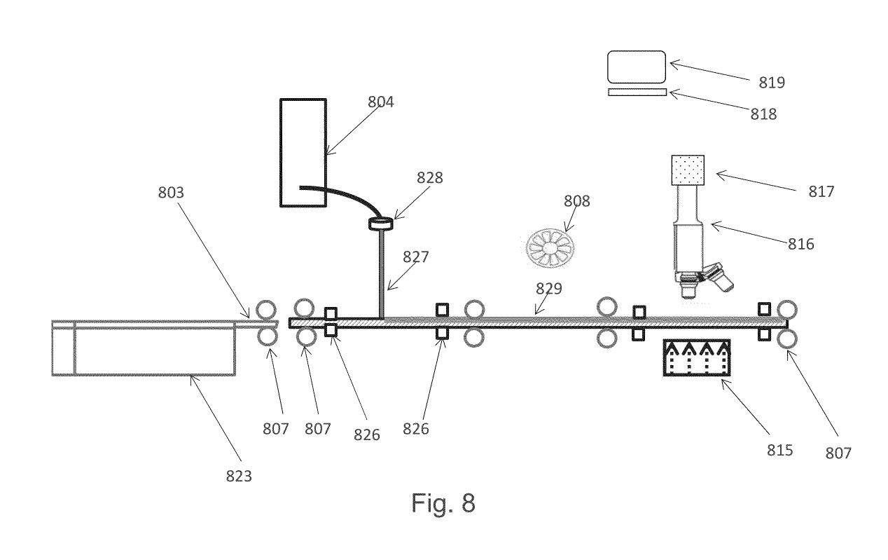

FIG. 8 illustrates schematically an example blood count apparatus for point of care testing. An asymmetric capillary tube 827 can be coated with a chemical, dye, a fluorescent antibody, or any other compound. For example, capillary tube 827 can be pre-coated with EDTA and methylene blue. Capillary tube 827 is then filled with a sample, e.g., whole blood. The larger inner diameter opening of the asymmetric capillary tube is used to pull the blood into the capillary tube. A small magnetic “flea” (not shown), as part of the sample applicator, can be required to mix the blood with the anti-coagulant and dye. The larger end of the asymmetric capillary tube is then manually or automatically inserted into collar 828. Pre-cut hydrophilic flexible polyester ribbon 803 is removed from cassette 823 by operation of ribbon controller rollers 807, and the hydrophilic surface can be positioned under capillary tube 827 with ribbon controller rollers 807 and guides 826. Ribbon controller guides 826 adjust the tension of ribbon 803, and the capillary tube 827 is brought down so the tip just touches the ribbon. Pump 804 dispenses the blood onto ribbon 803 at a dispense rate of, for example, 0.04 μl/sec while ribbon controller guides the ribbon at a substantial constant velocity of, for example, 35 mm/sec. It should be appreciated, however, that any other dispensing rate and velocity can be utilized.

In one embodiment, a single column of cells 829 can be about 14-18 cm long and about 0.4 mm to about 0.5 mm wide, and can be laid down in about 7.5 seconds. However, it should be appreciated that the above parameters are described by way of example only, and any other dimensions of the cell column and the speed with which it is formed can be utilized. The column of cells can be dried with fan 808, and the entire stained column of cells 829 can be brought through the optical axis of a light receiver 816 at a magnification, e.g., of 40×.

A LED light source 815 can expose the sample 829 to light at a first wavelength of, for example, 430 nm. A first composite image can be formed. All parameters associated with a complete blood count can be determined using computer software stored in at least one tangible, non-transitory, computer-readable storage medium included in computer 818 associated with display 819. Abnormal cells can be identified automatically (e.g., using pattern recognition technique(s)) by the computer software and/or can be identified manually by an operator. It should be appreciated that the acquired information on the cells can be analyzed in any suitable manner.

Rollers 807 can reverse direction, and a filter (not shown for the sake of simplicity) can be applied to LED light source 815, and/or LED light source 815 is rotated. Rollers 807 reverse direction to advance the flexible ribbon under the light receiver. The sample is exposed to light at a second wavelength of, for example, 500 nm. A second composite image can then be formed. All parameters associated with a complete blood count can be determined using the computer software stored in the at least one tangible, non-transitory, computer-readable storage medium in computer 818. Abnormal cells can be identified by the user, or computer. Rollers 807 reverse direction, and LED light source may be modified twice more to expose sample to light at third and fourth wavelengths of 575 nm and 600 nm. Separate third and fourth composite images can be formed and parameters associated with a complete blood count can be determined using software stored inside computer 818. Abnormal cells can be identified by the operator, or computer. Following analysis, ribbon 803 can be sent to a waste bin (not shown) via ribbon controller 807, and capillary tube 827 is removed from the instrument, and discarded.

A process of some embodiments can comprise one or more of the following steps: positioning a tip just touching the ribbon; dispensing diluted cells out of the tip at a rate of about 0.05 uL per second; and moving the ribbon at speed of 16 mm per second. In other embodiments, the process includes one or more of the steps: providing the system with new ribbon stored as roll; providing a first station where the cells are dispensed onto the ribbon in a monolayer; providing a second optional station for fixing, staining, and drying the cells spread on the ribbon; providing a third station for capturing the images of the cells on the ribbon; optionally providing slides to which the ribbon can be fixed and collecting said slides; and collecting the used ribbon on a collector wheel.

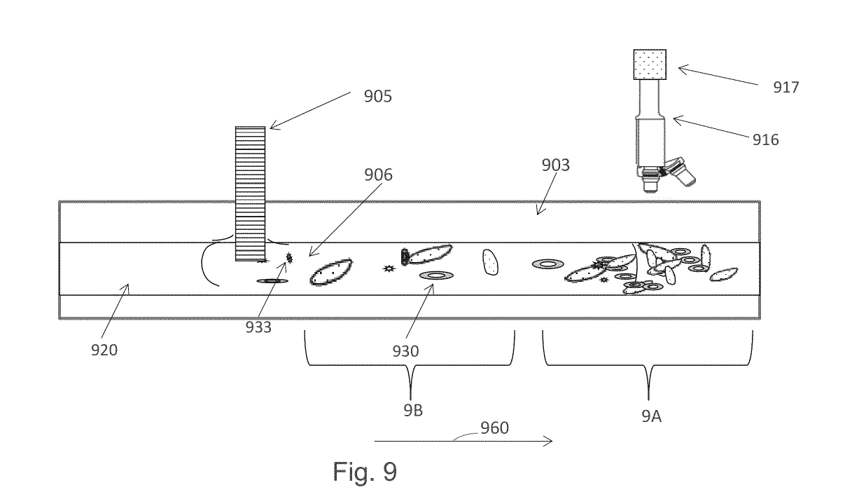

FIG. 9 illustrates a sample applicator 905 that can deposit various cells 930 of various types on a hydrophilic surface 920 of flexible ribbon 903 being pulled in the direction of arrow 960 by a ribbon controller (not shown) to light receiver 916. Light receiver 916 can also include a camera 917, and can be coupled with a computer storing and executing imaging software (e.g., computer 118 in FIG. 1), which is not shown for the sake of simplicity. As schematically shown in FIG. 9, an initial sample 9A which does not have a monolayer of cells can be dispensed on to ribbon 903. If the imaging software executed by a processor does not detect a monolayer of cells, the computer can instruct the ribbon controller to increase the velocity of the ribbon to form a monolayer of cells. Alternatively, the computer can instruct sample applicator 905 to decrease the volume of sample dispensed—in this way, a monolayer of cells indicated as 9B can be formed. Monolayer of cells 9B can then be deposited on to flexible ribbon 903 having hydrophilic surface 120.

Alternatively or additionally, if the imaging software detects an excessive amount of “white space” on the hydrophilic surface, the computer can instruct the ribbon controller to decrease the velocity of the ribbon and/or instruct the sample applicator to increase the volume dispensed.

In one embodiment, the single column of cells having a width of approximately 0.4 mm to approximately 0.75 mm can be deposed on a hydrophilic surface of a flexible ribbon having a width 3-15 mm. The ribbon can be wide enough to be driven on the outside by rollers 906 pressing on the top and underside of the ribbon. This ribbon can make a single pass through the optical axis of a microscope or multiple passes when different objectives are required to increase the magnification. Multiple passes through the optical axis of a microscope can also be used when different wavelengths of lights contained in the light source are used to illuminate stained the cells. The width of columns of cells can vary (e.g., between 0.1 and 2.0 mm wide) depending on the testing required. For example, a complete blood count can require a narrower column than a five part differential test.

In some embodiments, the described techniques can be used to perform blood cell morphology analysis, a 5-part differential analysis, or any other type of analyses. The described techniques can allow depositing a large number of cells on a substrate (e.g., a flexible ribbon) for analysis, which cannot be possible to achieve using glass slides. In some embodiments, a long (e.g., at least 200 meters) piece of flexible ribbon can be used to dispense cells thereon in a monolayer, and a variety of different tags (e.g., fluorescent or other type) can be applied to the cells. The subsequent analysis of such cells can be done with an improved speed—e.g., less than an hour. Furthermore, because a microscope can need to move only in one direction, the simplicity and speed of the analysis can be improved.

Having thus described several aspects of at least one embodiment of this invention, it is to be appreciated that various alterations, modifications, and improvements will readily occur to those skilled in the art.

The embodiments and examples presented herein are illustrative of the general nature of the subject matter claimed and are not limiting. It can be understood by those skilled in the art how these embodiments can be readily modified and/or adapted for various applications and in various ways without departing from the spirit and scope of the subject matter disclosed claimed. The claims hereof are to be understood to include without limitation all alternative embodiments and equivalents of the subject matter hereof. Phrases, words and terms employed herein are illustrative and are not limiting. It should be appreciated that any aspects of the different embodiments disclosed herein can be combined in a range of possible alternative embodiments, and alternative combinations of features, all of which varied combinations of features are to be understood to form a part of the subject matter claimed.

What is claimed is:

1. A microscopy system, comprising:
a sample applicator having a tip end configured to dispense a sample;
a flexible ribbon having a hydrophilic surface configured to receive a sample from the tip;
a light receiver; and
a ribbon controller configured to receive the flexible ribbon and guide the ribbon under the light receiver.

2. The system of claim 1, wherein the flexible ribbon is formed from a material selected from the group consisting of a polymer, polyester, polystyrene, and a co-polymer.

3. The system of claim 1, wherein the flexible ribbon is optically clear in the range of about 400 nm to 700 nm.

4. The system of claim 1, wherein the ribbon controller is configured to guide the ribbon under the light receiver at a substantially constant velocity.

5. The system of claim 1, wherein the light receiver comprises at least one lens.

6. The system of claim 1, wherein the light receiver comprises a magnifying lens.

7. The system of claim 1, wherein the light receiver comprises a camera.

8. The system of claim 1, wherein the flexible ribbon has a thickness in the range of about 0.04 mm to 1.0 mm, and a width in the range of about 2.5 mm to 30 mm.

9. The system of claim 1, wherein the flexible ribbon has a length in the range of about 10 cm to 100,000 cm.

10. The system of claim 1, further comprising a roll for dispensing the ribbon, wherein the ribbon controller is configured to receive the flexible ribbon from the roll.

11. The system of claim 1, further compromising a mechanism for cutting the ribbon into a plurality or pieces, wherein the ribbon controller is configured to receive at least one piece of the plurality of pieces.

12. The system of claim 11, further comprising a collector for receiving the pieces of ribbon following passage under the light receiver.

13. The system of claim 1, wherein the sample applicator is configured to dispense a monolayer of cells on the hydrophilic surface of ribbon.

14. The system of claim 1, wherein the sample applicator comprises an applicator pump.

15. The system of claim 1, further comprising:
a diluent vessel in operable communication with the sample applicator, wherein the diluent vessel comprises at least one diluent; and
a diluent pump configured to deliver at least one diluent to the sample applicator.

16. A method for preparing a sample for microscopy, the method comprising:
engaging a flexible piece of ribbon having a hydrophilic surface with at least one ribbon controller;
dispensing cells from a tip of a sample applicator comprising the sample on to the hydrophilic surface; and
guiding the flexible ribbon under a dispenser configured to dispense fixing and/or staining solution using the at least one ribbon controller.

17. The method of claim 16, wherein the dispenser is configured to use methanol and Wright Giemsa stain.

18. The method of claim 16, wherein the flexible ribbon is optically clear in the range of about 400 nm to about 700 nm.

19. The method of claim 16, further comprising guiding the flexible ribbon with at least one ribbon controller under a light receiver, wherein the light receiver is configured to analyze cells.

20. The method of claim 19, wherein the light receiver comprises a camera that is configured to acquire images of the cells.

21. The method of claim 16, wherein the sample applicator comprises an applicator pump that dispenses cells from the tip of the sample applicator.

22. The method of claim 16, further comprising using a storage vessel having at least one diluent in operable communication with the sample applicator, and a diluent pump that delivers the at least one diluent to the sample applicator.

23. The method of claim 16, wherein the sample applicator dispenses a monolayer of cells on to the hydrophilic surface.

24. The method of claim 16, further comprising executing computer-executable instructions stored on at least one tangible, non-transitory computer-readable storage medium such that the computer-executable instructions, when executed by at least one processor, cause a computing device to instruct the sample applicator to vary the distance between the tip of the sample applicator and the flexible ribbon.

25. The method of claim 16, further comprising:
executing computer-executable instructions stored on at least one tangible, non-transitory computer-readable storage medium such that the computer-executable instructions, when executed by at least one processor, cause a computing device to perform at least one of:
instructing the sample applicator to dispense a sample on to the hydrophilic surface;
instructing a diluent vessel to dilute or not dilute the sample;
instructing the at least one ribbon controller to vary the distance between the ribbon and the tip of the sample applicator;
instructing the at least one ribbon controller to alter a tension of the ribbon;
instructing the at least one ribbon controller in what direction to guide the ribbon;
instructing the at least one ribbon controller to move the ribbon at a specified velocity, and/or change the velocity of the ribbon relative to a light receiver;
instructing the light receiver to adjust focus in response to a signal received by the light receiver;
instructing the sample applicator to dispense the sample at a specific rate; and
instructing the sample applicator to dispense the sample at a specific volume.

26. A method for analyzing cells, comprising:
engaging a portion of a flexible ribbon having a hydrophilic surface with at least one ribbon controller;
dispensing cells from a tip of a sample applicator comprising a sample of the cells on the hydrophilic surface;
guiding the portion of the flexible ribbon under a dispenser configured to dispense fixing and/or staining solution using the at least one ribbon controller; and
guiding the flexible ribbon with at least one ribbon controller under a light receiver, wherein the light receiver is configured to analyze cells.

27. The method of claim 26, wherein the flexible ribbon is guided under the light receiver at a substantially constant velocity.

28. The method of claim 26, wherein the flexible ribbon is optically clear in the range of about 400 nm to about 700 nm.

29. The method of claim 26, wherein the light receiver comprises a camera configured to acquire images of cells.

30. The method of claim 26, wherein the sample applicator comprises an applicator pump configured to dispense cells from the tip of the sample applicator.

31. The method of claim 26, further comprising using a storage vessel having at least one diluent in operable communication with the sample applicator, and a diluent pump that delivers the at least one diluent to the sample applicator.

32. The method of claim 26, wherein the sample applicator dispenses a monolayer of cells on to the hydrophilic surface.

33. The method of claim 26, further comprising executing computer-executable instructions stored on at least one tangible, non-transitory computer-readable storage medium such that the computer-executable instructions, when executed by at least one processor, cause a computing device to instruct the sample applicator to vary the distance between the tip of the sample applicator and the flexible ribbon.

34. The method of claim 26, further comprising:
executing computer-executable instructions stored on at least one tangible, non-transitory computer-readable storage medium such that, the computer-executable instructions, when executed by at least one processor, cause a computing device to perform at least one of:
instructing the sample applicator to dispense the sample on to the hydrophilic surface;
instructing a diluent vessel to dilute or not dilute the sample;
instructing the at least one ribbon controller to vary the distance between the ribbon and the tip of the sample applicator;
instructing the at least one ribbon controller to alter a tension of the ribbon;
instructing the at least one ribbon controller in what direction to guide the ribbon;
instructing the at least one ribbon controller to move the ribbon at a specified velocity, and/or change the velocity of the ribbon relative the light receiver;
instructing the at least one light receiver to adjust focus in response to a signal received by the light receiver;
instructing the sample applicator to dispense the sample at a specific rate; and
instructing the sample applicator to dispense the sample at a specific volume.

35. The method of claim 26, further comprising acquiring a plurality of images of the cells using the light receiver, and analyzing the plurality of images to obtain a 5-part differential.

36. The method of claim 26, wherein the dispenser is configured to use methanol and Wright Giemsa stain.

37. A kit, comprising:
a flexible ribbon having a hydrophilic surface configured to receive a biological sample, wherein the flexible ribbon is formed from a material selected from the group consisting of a polyester, polystyrene, and mixtures thereof, and wherein the flexible ribbon is optically clear in the range of about 400 nm to about 700 nm; and
instructions for installing the ribbon on a microscopy system comprising a sample applicator having a tip end, a light receiver, and at least one ribbon controller to receive and guide the ribbon under the light receiver.

38. A system for analyzing cells from blood, bone marrow or cell culture comprising:
an applicator comprising a tip adapted for applying a single flow of cells in a monolayer on a glass substrate.
a light or image receiving device adapted for capturing images and fluorescence from the cells on the glass substrate; and
a computer adapted for
instructing the system to aspirate a sample from a collection vessel,
instructing the system to position the tip just above the glass substrate,
instructing the system to move the glass substrate at a predetermined velocity, and
instructing the applicator to dispense the sample at a predetermined rate such that a monolayer of cells results with a width that is between 0.6 and 2.0 mm and a length that is between 6 and 12 cm for a 5 part differential white cell count.
